(19) United States
(12) Patent Application Publication
(10) Pub. No.: US 2022/0096682 A1
(43) Pub. Date: Mar. 31, 2022

(54) VEHICLE IGNITION INTERLOCK DECONTAMINATION AND CALIBRATION STATION

(71) Applicant: Best Labs Leasing, LLC, North Lima, OH (US)

(72) Inventors: Nicholas G. Doinoff, North Lima, OH (US); Eugene P. O'Dea, Girard, OH (US); Neal A. Wire, Struthers, OH (US)

(21) Appl. No.: 17/487,884

(22) Filed: Sep. 28, 2021

Related U.S. Application Data
(60) Provisional application No. 63/085,514, filed on Sep. 30, 2020.

(51) Int. Cl. A61L 2/10

(52) U.S. Cl. CPC A61L 2/10; G01N 33/4972

(57) ABSTRACT

A decontamination and calibration station for vehicle ignition interlock devices that includes a self-contained structure that receives and encloses an interlock device operatively connected thereto. The station includes at least one calibration reference standard operatively connected to a valve assembly of the station, which is fluidly and operatively connected to an interior of the station and the interlock device for calibration of the device. The station includes a decontamination feature to sanitize and disinfect the station and/or the interlock device. The decontamination feature can include at least one UV-C bulb fixture mounted in the station and/or disinfectant operatively connected to the station and interlock device. The station includes a control system for controlling calibration of the interlock device and the decontamination feature, which can be a computer operatively connected directly to the interlock device and the station or a processor integrated into the station controlled by a remote device.

### CROSS-REFERENCE TO RELATED APPLICATION

This application claims the benefit of U.S. Provisional Patent Application Ser. No. 63/085,514, filed on Sep. 30, 2020.

### BACKGROUND

### Technical Field

The disclosed subject matter relates generally to ignition interlock devices or breath alcohol detection ignition interlock devices for vehicles. More specifically, the disclosed subject matter is directed to a decontamination and calibration station for vehicle ignition interlock devices. The vehicle ignition interlock device decontamination and calibration station of the disclosed subject matter includes a self-contained structure that enables a vehicle ignition interlock device to be operatively connected to the decontamination and calibration station within the structure via a modular cable and calibration tube of the station. The decontamination and calibration station includes a high-pressure purge feature that ensures accurate calibration of the vehicle ignition interlock device and a clean sample chamber of the interlock device subsequent to calibration. The decontamination and calibration station includes a sanitization and disinfecting feature or decontamination feature that employs UV-C light bombardment or a disinfecting aerosol to sanitize and disinfect the interior of the decontamination and calibration station and the ignition interlock device prior to returning the device to a vehicle operator for use. The decontamination and calibration station enables a vehicle ignition interlock device to be both calibrated and disinfected and/or sanitized with minimal interaction with the interlock device via a program for controlling and/or monitoring the processes, which is optionally operated by a remote computer or a computer integrated into the station, such as a simple touch screen interface operatively connected to the decontamination and calibration station and interlock device.

### Background Art

Vehicle ignition interlock devices have been utilized for many years. A vehicle ignition interlock device is connected to a vehicle's ignition system and requires a vehicle operator to blow into a mouthpiece of the interlock device to measure the operator's breath alcohol or ethanol concentration before starting or continuing operation of a vehicle. If the vehicle operator's breath alcohol concentration analyzed by the device is greater than a pre-programmed blood alcohol concentration limit, the interlock device interrupts the signal from the vehicle ignition to the vehicle starter and prevents the engine from being started. Alternatively, if the driver is currently operating the vehicle and the operator's breath alcohol concentration is greater than the pre-programmed blood alcohol concentration limit, the vehicle ignition interlock device will log the event, warn the driver, and then start an alarm in accordance with state regulations until the ignition is turned off or a passing breath alcohol sample is provided.

Modern vehicle ignition interlock devices are analytical devices that typically utilize an alcohol-specific fuel cell as the sensor, which is in communication with the mouthpiece of the interlock device via tubing, to analyze a vehicle operator's breath alcohol concentration. The fuel cell is an electrochemical device in which alcohol undergoes a chemical oxidation at a catalytic electrode surface to generate an electric current. The current in turn is measured and converted to a blood alcohol concentration equivalent reading by the vehicle ignition interlock device.

Like most analytical devices, the vehicle ignition interlock device must be calibrated periodically, typically at 30, 60, or 90 day intervals, to ensure accuracy of a vehicle operator's breath alcohol concentration provided by the interlock device. Periodic calibration of the vehicle ignition interlock device is often performed using a discrete calibration system that is connectable to the vehicle ignition interlock device and utilizes an alcohol reference standard of either a pressurized alcohol-gas mixture of a known concentration, or an alcohol wet bath arrangement that contains an alcohol solution with a known concentration, which is introduced into the fuel cell via the calibration system or is manually introduced into the fuel cell. Such calibrations are typically performed by an authorized technician at a remote calibration site, which typically requires the vehicle operator either to send the vehicle ignition interlock device to the remote site or to travel to the site for the calibration to be performed.

While such vehicle ignition interlock device calibration systems and processes are typically suitable for their intended purpose, they often require considerable interaction between the technician and the device to perform the calibration, which increases the potential for errors in the calibration process, and thus potentially inaccurate calibration of the device. Moreover, with such calibration systems and processes, the technician often simply introduces an alcohol calibration reference standard into the vehicle ignition interlock device fuel cell without employing adequate processes and procedures to ensure that the device is clean prior to calibration, which can result in an inaccurate calibration. Such calibration systems and processes also do not include any sanitizing and disinfecting procedures to ensure that the entire interlock device, including the internal components of the device, such as the interior of the mouthpiece, tubing, fuel cell, and/or exhaust tubing, are adequately sanitized and disinfected prior to being returned to a vehicle operator for use.

Thus, a need exists in the art for a vehicle ignition interlock device decontamination and calibration station that includes a self-contained structure that enables a vehicle ignition interlock device to be operatively connected to the station within the structure. There is also a need in the art for a vehicle ignition interlock device decontamination and calibration station that includes a feature that ensures accurate calibration of the vehicle ignition interlock device and a clean sample chamber of the interlock device subsequent to calibration. There is also a need in the art for a vehicle ignition interlock device decontamination and calibration station that includes a sanitization and disinfecting feature to sanitize and disinfect the interior of the decontamination and calibration station and the interlock device prior to returning the device to a vehicle operator for use. There is also a need in the art for a vehicle ignition interlock device decontamination and calibration station that enables an interlock device to be both calibrated and disinfected and/or sanitized with minimal interaction with the interlock device. The vehicle ignition interlock device decontamination and calibration station of the disclosed subject matter satisfies these needs and will now be described.

### BRIEF DESCRIPTION OF THE DISCLOSED SUBJECT MATTER

An objective of the disclosed subject matter is to provide a decontamination and calibration station for vehicle ignition interlock devices with a self-contained structure that enables a vehicle ignition interlock device to be operatively connected to the decontamination and calibration station enclosed within the structure.

Another objective of the disclosed subject matter is to provide a decontamination and calibration station for vehicle ignition interlock devices that ensures accurate calibration of a vehicle ignition interlock device and provides the interlock device with a clean sample chamber subsequent to calibration.

Yet another objective of the disclosed subject matter is to provide a decontamination and calibration station for vehicle ignition interlock devices that includes a sanitization and disinfecting feature to disinfect and sanitize the decontamination and calibration station and/or the vehicle ignition interlock device prior to returning the device to a vehicle operator for use.

Another objective of the disclosed subject matter is to provide a decontamination and calibration station for vehicle ignition interlock devices that enables a vehicle ignition interlock device to be calibrated and disinfected and/or sanitized with minimal interaction with the interlock device.

These objectives and other are achieved by the vehicle ignition interlock device decontamination and calibration station of the disclosed subject matter, which includes a housing, the housing being configured to receive and enclose a vehicle ignition interlock device within an interior of the housing; a valve assembly operatively and fluidly connected with the interior of the housing, the valve assembly being further operatively and fluidly connected to the vehicle ignition interlock device received and contained within the interior of the housing; at least one reference standard being operatively connected to the valve assembly, the at least one reference standard enabling the ignition interlock device to be calibrated; a decontamination feature integrated into the decontamination and calibration station, the decontamination feature enabling the decontamination and calibration station and/or the vehicle ignition interlock device received and contained in the interior of the housing to be sanitized and/or disinfected; and a control system, the control system enabling actuation of the valve assembly and operation of the decontamination feature.

These objectives and other are also achieved by a method for decontaminating and calibrating a vehicle ignition interlock device of the disclosed subject matter, which includes the steps of providing a decontamination and calibration station for a vehicle ignition interlock device; enclosing the vehicle ignition interlock device within the decontamination and calibration station; decontaminating the vehicle ignition interlock device via the decontamination and calibration station; and calibrating the vehicle ignition interlock device with the decontamination and calibration station.

### BRIEF DESCRIPTION OF THE SEVERAL VIEWS OF THE DRAWINGS

An exemplary embodiment of the disclosed subject matter, illustrative of the best mode in which Applicant has contemplated applying the principles, is set forth in the following description and is shown in the drawing.

FIG. 1 is a perspective view of an exemplary embodiment vehicle ignition interlock device decontamination and calibration station of the disclosed subject matter; and

FIG. 2 is a fragmentary perspective view of the exemplary embodiment vehicle ignition interlock device decontamination and calibration station shown in FIG. 1, showing operative and fluid connection of a valve assembly of the decontamination and calibration station to a housing of the station via a pair of hoses.

Similar numerals refer to similar parts throughout the drawing.

### DETAILED DESCRIPTION OF THE DISCLOSED SUBJECT MATTER

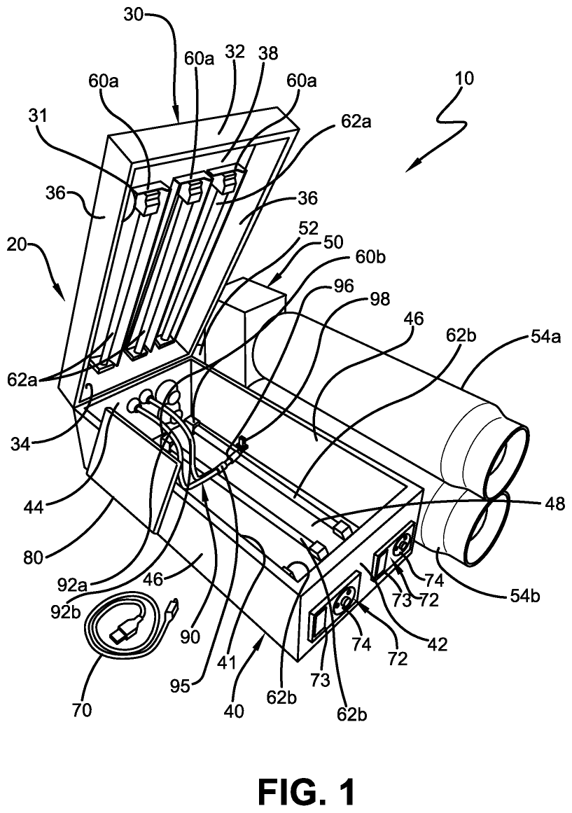

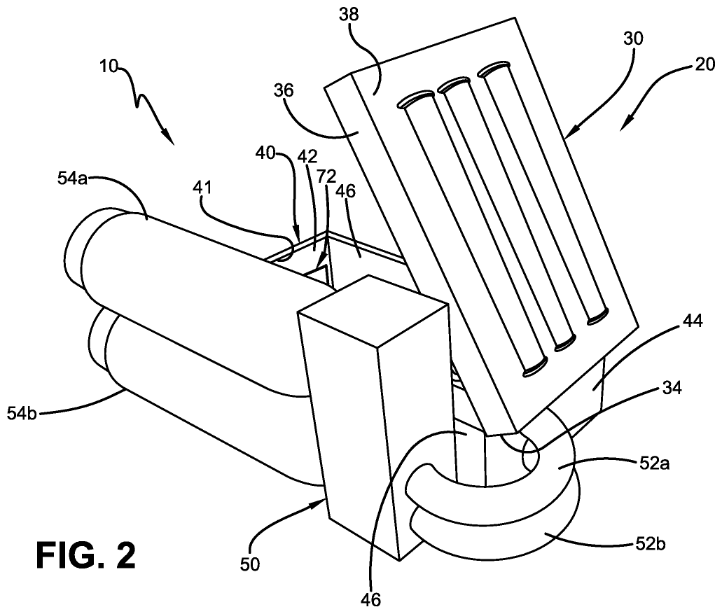

An exemplary embodiment vehicle ignition interlock device decontamination and calibration station of the disclosed subject matter is shown in FIGS. 1 and 2, and is indicated generally at reference numeral 10. With reference to FIGS. 1-2, decontamination and calibration station 10 includes a two-part housing 20, which includes a top portion or lid 30 and a bottom portion 40. Top portion 30 is formed of a suitable rigid material and has a generally rectangular box-like structure including a front wall 32 (FIG. 1) and a rear wall 34. Top portion 30 further includes a pair of side walls 36, each one of which is rigidly connected to an opposite side of front wall 32 (FIG. 1) and rear wall 34 by any suitable means, such as welds or fasteners, so that they extend generally parallel to one another. Front wall 32 (FIG. 1), rear wall 34, and side walls 36 are rigidly connected to a top wall 38 of top portion 30 by any suitable means, such as welds or fasteners. Alternatively, front wall 32 (FIG. 1), rear wall 34, side walls 36, and top wall 38 could be integrally formed with one another.

Bottom portion 40 of housing 20 is formed of a suitable rigid material and has a generally rectangular box-like structure that corresponds generally to the width and length of top portion 30. Bottom portion 40 includes a front wall 42 and a rear wall 44. Bottom portion 40 further includes a pair of side walls 46, each one of which is rigidly connected to an opposite side of front wall 42 and rear wall 44 by any suitable means, such as welds or fasteners, so that they extend generally parallel to one another. Front wall 42, rear wall 44, and side walls 46 are rigidly connected to a bottom wall 48 (FIG. 1) of bottom portion 40 by any suitable means, such as welds or fasteners. Alternatively, front wall 42, rear wall 44, side walls 46, and bottom wall 48 (FIG. 1) could be integrally formed with one another.

Top portion 30 of housing 20 is pivotally connected to bottom portion 40 via suitable means, such as a hinged connection (not shown). In this manner, housing 20 can be opened by upward arcuate movement of top portion 30 relative to bottom portion 40 about the pivotal connection to the bottom portion to enable access to an interior space 31 (FIG. 1) of the top portion and an interior space 41 of the bottom portion. Housing 20 can be closed by positioning top portion 30 adjacent bottom portion 40 such that the bottom surfaces of front wall 32 (FIG. 1), rear wall 34, and side walls 36 of the top portion rest on the top surfaces of front wall 42, rear wall 44, and side walls 46 of the bottom portion to prevent access to interior spaces 31 FIG. 1) and 41. It is to be understood that housing 20 of exemplary embodiment vehicle ignition interlock device decontamination and calibration station 10, could have different shapes and/or configurations than that shown and described without affecting the overall concept or operation of the disclosed subject matter.

With particular reference to FIG. 1, top portion 30 of housing 20 includes a plurality of UV-C bulb fixtures 60a mounted on the bottom surface of top wall 38, such that the bulb fixtures extend longitudinally along the bottom surface of the top wall. Each fixture 60a enables mounting and operative connection of a UV-C bulb 62a. Although top portion 30 is shown with three (3) UV-C bulb fixtures 60a mounting and operatively connecting three (3) UV-C bulbs 62a, it is to be understood that the top portion could include more or less than three UV-C bulb fixtures with corresponding UV-C bulbs without affecting the overall concept or operation of the disclosed subject matter.

Bottom portion 40 of housing 20 also includes a plurality of UV-C bulb fixtures 60b mounted on the top surface of bottom wall 48, such that the bulb fixtures extend longitudinally on the top surface of the bottom wall. Each fixture 60b enables mounting and operative connection of a UV-C bulb 62b. Although bottom portion 40 is shown with three (3) UV-C bulb fixtures 60b mounting and operatively connecting three (3) UV-C bulbs 62b, it is to be understood that the bottom portion could also include more or less than three UV-C bulb fixtures with corresponding UV-C bulbs without affecting the overall concept or operation of the disclosed subject matter.

With continued reference to FIG. 1, exemplary embodiment vehicle ignition interlock device decontamination and calibration station 10 further includes a pair of filtered exhaust fan assemblies 72. Each fan assembly 72 includes a fan housing 73, which are mounted within respective openings (not shown) formed in front wall 42 (FIG. 1-2) of bottom portion 40 of housing 20. Each fan assembly 72 mounts a respective filter (not shown) and exhaust fan 74, the importance of which will be described in detail below.

With reference to FIGS. 1-2, exemplary embodiment vehicle ignition interlock device decontamination and calibration station 10 further includes a valve assembly 50. Valve assembly 50 includes a generally cuboidal shape and is attached to the outside surface of one of side walls 46 of bottom portion 40 of housing 20. Valve assembly 50 includes a pair of vertically aligned threaded openings (not shown). The threaded openings of valve assembly 50 enable attachment of two alcohol calibration reference standard canisters 54a and 54b containing respective pressurized alcohol calibration reference standard gases of known concentrations, which threadably engage the openings to connect the canisters to the valve assembly. It is to be understood that valve assembly 50 could be detached from bottom portion 40 of housing 20 without affecting the overall concept or operation of the disclosed subject matter.

It is contemplated that canisters 54a and 54b could contain pressurized alcohol calibration reference standard gas with the same concentrations, with only one of the gasses being utilized to calibrate a vehicle ignition interlock device when only a single point calibration of the vehicle ignition interlock device is desired. It is also contemplated that canisters 54a and 54b could contain pressurized alcohol calibration reference standard gasses with different concentrations, for example, to perform a two-point calibration of the vehicle ignition interlock device or to accommodate single-point calibrations of various vehicle ignition interlock devices with different desired pre-programmed blood alcohol concentration limits. It is further contemplated that one of canisters 54a or 54b could be replaced with a canister containing pressurized aerosolized disinfectant, the importance of which will be described below.

Valve assembly 50 houses a pair of solenoid valves (not shown), with each valve being in communication with a respective canister 54a, 54b. Valve assembly 50 is operatively and fluidly connected to housing 20. More specifically, valve assembly 50 includes a pair of hoses 52a and 52b (FIG. 1) that are connected to valve assembly 50 such that they are in fluid communication with a respective solenoid valve housed within the valve assembly. Each hose 52 in turn is connected to a respective opening (not shown) formed in rear wall 44 of bottom portion 40 of housing 20.

Exemplary embodiment vehicle ignition interlock device decontamination and calibration station 10 includes a calibration tube assembly 90 for operatively and fluidly connecting an ignition interlock device (not shown) to the decontamination and calibration station. More specifically, calibration tube assembly 90 includes a pair of tubes 92a and 92b that are each connected to a respective one of the openings formed in rear wall 44 (FIGS. 1-2) of bottom portion 40 of housing 20. In this manner, each one of tubes 92a and 92b is in fluid communication with a respective one of pair of hoses 52a and 52b (FIGS. 1-2) of valve assembly 50, and thus a respective canister 54a and 54b. Tubes 92a and 92b are connected to a Y-shaped connector 95, which in turn is connected to a vehicle ignition interlock device connector 96. Connector 96 includes a port 98, which is connectable with a mouthpiece (not shown) of a vehicle ignition interlock device (not shown) to enable pressurized alcohol reference standard gas from canisters 54a and/or 54b to be introduced into the interlock device during calibration, as will be described in detail below.

Exemplary embodiment vehicle ignition interlock device decontamination and calibration station 10 includes a computer 80 with a program installed thereon for controlling functionality of the various components of the decontamination and calibration station and a connected vehicle ignition interlock device. Computer 80 is mounted to one of side walls 46 (FIGS. 1-2) of bottom portion 40 of housing 20 by any suitable means. Computer 80 is operatively and electronically connected to valve assembly 50, fan assemblies 72, and UV-C bulb fixtures 60a and 60b by any suitable means, for example, via wiring or RF communication, such that the computer is capable of controlling operation of the solenoid valves within the valve assembly, exhaust fans 74 of the exhaust fan assemblies, and UV-C bulbs 62a and 62b of the UV-C bulb fixtures.

Computer 80 is also operatively connectable to a vehicle ignition interlock device (not shown). More specifically, exemplary embodiment vehicle ignition interlock device decontamination and calibration station 10 includes a modular data cable 70, which is connectable to both the vehicle ignition interlock device and computer 80 via a port (not shown) formed in the side wall 46 (FIGS. 1-2) of bottom portion 40 (FIGS. 1-2) to which the computer is also attached. Data cable 70 is connectable with various vehicle ignition interlock devices from different manufactures. In this manner, data cable 70 enables communication between decontamination and calibration station 10 and the vehicle ignition interlock device to enable the computer to control calibration of the vehicle ignition interlock device with minimal interaction with the interlock device via the program installed on computer 80. It is contemplated that exemplary embodiment vehicle ignition interlock device decontamination and calibration station 10 could employ other means of communication between the station and/or computer 80 and the vehicle interlock device than data cable 70, such as via cellular or Wi-Fi communication with vehicle interlock devices capable of such communication, without affecting the overall concept or operation of the disclosed subject matter. As shown, computer 80 is a tablet-like device that includes a user-friendly touch screen that enables a technician to control the calibration of the connected vehicle ignition interlock device, but could have other structures, components, or features that enable control of the calibration of the vehicle ignition interlock device without affecting the overall concept or operation of the disclosed subject matter.

Alternatively, exemplary embodiment vehicle ignition interlock device decontamination and calibration station 10 could include means other than computer 80 for controlling functionality of the various components of the decontamination and calibration station and a connected vehicle ignition interlock device without affecting the overall concept or operation of the disclosed subject matter. For example, decontamination and calibration station 10 could have a processor integrated into the station that includes a program for controlling functionality of the of the various components of the decontamination and calibration station. The program can optionally be controllable from a remote device, such as a smart phone or a remote computer.

Having described the structure and various components of exemplary embodiment vehicle ignition interlock device decontamination and calibration station 10, the operation the decontamination and calibration station will now be described. In order to calibrate a vehicle ignition interlock device (not shown) with decontamination and calibration station 10, the interlock device is first operatively connected to the station by simply connecting data cable 70 to a data port on the interlock device and connecting a mouthpiece (not shown) of the interlock device to port 98 of vehicle ignition interlock device connector 96. When connected to decontamination and calibration station 10 in the manner described above, the vehicle ignition interlock device is positioned within interior space 41 of bottom portion 40 of housing 20 (FIGS. 1-2). Top portion 30 of housing 20 is then closed. Depending on the size of the vehicle ignition interlock device, the interlock device can also extend into interior space 31 of top portion 30 of housing 20 (FIGS. 1-2). In this manner, the vehicle ignition interlock device is completely enclosed within housing 20 (FIGS. 1-2) of decontamination and calibration station 10. Once the vehicle ignition interlock device is operatively connected to decontamination and calibration station 10 and enclosed within housing 20 (FIGS. 1-2) of the station, a technician or operator controls the calibration of the interlock device via computer 80.

In accordance with an important aspect of exemplary embodiment vehicle ignition interlock device decontamination and calibration station 10, and with reference to FIGS. 1-2, the decontamination and calibration station includes a high-pressure purge feature that enables the vehicle ignition interlock device to be purged with high-pressured alcohol reference standard gas from canister 54a and/or 54b. More specifically, a technician can select a “purge” command via computer 80 (FIG. 1), which in turn relays a signal to a selected solenoid valve in fluid communication with the associated canister 54a or 54b. In response to the signal received, the selected solenoid valve opens to allow the alcohol reference standard gas to flow at a pre-determined pressure from the associated canister 54a or 54b, through the respective valve, hose 52a or 52b, tube 92a or 92b (FIG. 1), Y-shaped connector 95 (FIG. 1), vehicle ignition interlock device connector 96 (FIG. 1), and ultimately into and through the vehicle ignition interlock device, including the fuel cell of the interlock device. The alcohol reference standard gas is exhausted from the vehicle ignition interlock device into interior spaces 31 and 41 of housing 20. The high-pressure purge feature can be pre-programmed to run for a selected period of time or can be manually terminated via a “stop purge” command or similar command on computer 80 (FIG. 1). The high-pressure purge feature ensures that the entire fluid path between canister 54a or 54b, and through the vehicle ignition interlock device, is flushed of any contaminates that may affect the accuracy of the calibration of the vehicle ignition interlock device, leaving only the unadulterated selected alcohol reference standard gas in the fuel cell of the vehicle ignition interlock device for calibration of the interlock device.

After the high-pressure purge of exemplary embodiment vehicle ignition interlock device decontamination and calibration station 10 and the connected vehicle ignition interlock device with the selected alcohol calibration reference standard gas from canister 54a or 54b, calibration of the interlock device is next performed. As the unadulterated selected alcohol reference standard gas from the selected canister 54a or 54b is already in the fuel cell via the high-pressure purge, a technician merely selects a “calibrate” command or similar command via computer 80 (FIG. 1), which in turn relays a signal to the vehicle ignition interlock device via operative connection to the interlock device by data cable 70 (FIG. 1) to calibrate the fuel cell of interlock device, the process of which is well known in the art. It is to be contemplated that the high-pressured “purge” command and “calibration” command could be pre-programmed as a single command, such as “purge-calibrate” on computer 80, without affecting the overall concept or operation of the disclosed subject matter. In this manner, decontamination and calibration station 10 ensures accurate calibration of the connected vehicle ignition interlock device.

In accordance with another important aspect of exemplary embodiment vehicle ignition interlock device decontamination and calibration station 10, and with reference to FIG. 1, the decontamination and calibration station includes a sanitization feature that enables the connected vehicle ignition interlock device and/or interior of housing 20 to be sanitized before and/or after calibration of the interlock device. Sanitization of the connected vehicle ignition interlock device and/or interior of housing 20 (FIGS. 1-2) is preferably by UV-C light bombardment. More specifically, a technician can select a “UV-C sanitize” command or similar command via computer 80, which in turn energizes UV-C bulb fixtures 60a and 60b and connected UV-C bulbs 62a and 62b. Once energized, UV-C bulbs 62a and 62b produce short wave radiation, typically between 100 nm and 280 nm, that is lethal to most bacteria, viruses, and/or other microorganisms. Moreover, UV-C bulbs 62a and 62b produce heat, which aids in sanitization of vehicle ignition interlock device decontamination and calibration station 10 and/or the connected vehicle ignition interlock device. The “UV-C sanitize” command can be pre-programmed to run the UV-C light bombardment for a selected period of time or can be manually terminated via a “stop sanitization” command or similar command on computer 80.

Thus, the exterior of all components within interior spaces 31 and 41 (FIGS. 1-2) of housing 20 of decontamination and calibration station 10, including the connected vehicle ignition interlock device, can be effectively disinfected and sanitized, without the use of cleaning solutions and solvents. Moreover, in components of decontamination and calibration station 10, such as calibration tube 90, and vehicle ignition interlock devices constructed of certain materials, the UV-C light can penetrate the materials, and thus penetrate the components of the station and/or the internal components of the interlock device, such as the tubing and fuel cell. This effectively sanitizes and disinfects both the exterior and interior of the components of decontamination and calibration station 10 and/or the exterior and internal components of the interlock device, ensuring that the station components and/or the interlock device are entirely sanitized and disinfected.

Alternatively, in certain vehicle ignition interlock devices with components that can be damaged by UV-C light bombardment, exemplary embodiment vehicle ignition interlock device decontamination and calibration station 10 could employ aerosolized disinfectant to sanitize and disinfect the interlock device. For example, and as indicated above, one of canisters 54a or 54b could be replaced with a canister containing pressurized aerosolized disinfectant. Computer 80 (FIGS. 1-2) could include a “sanitization purge” command or similar command selected by a technician, which in turn relays a signal to the selected solenoid valve in fluid communication with the canister containing the pressurized aerosolized disinfectant. In response to the signal received, the selected solenoid valve can open to allow the aerosolized disinfectant to flow at a pre-determined pressure from the canister, through the valve, hose 52a or 52b (FIGS. 1-2), tube 92a or 92b, Y-shaped connector 95, vehicle ignition interlock device connector 96, and ultimately into and through the vehicle ignition interlock device, including the tubing and fuel cell of the interlock device, thus sanitizing and disinfecting the internal components of the interlock device. Decontamination and calibration station 10 could also include means to introduce the aerosolized disinfectant into interior spaces 31 and 41 (FIGS. 1-2) of housing 20 to disinfect components of the decontamination and calibration station within the housing and/or the exterior of the connected vehicle ignition interlock device.

In accordance with yet another important aspect of exemplary embodiment vehicle ignition interlock device decontamination and calibration station 10, the decontamination and calibration station includes an exhaust feature that enables the interior of housing 20 and the internal components of the connected vehicle ignition interlock device, such as the tubing and the fuel cell, to be exhausted of all residual alcohol reference standard gas and/or aerosolized disinfectant, if applicable, after calibration and sanitization of the interlock device is complete. More specifically, a technician can select an “exhaust” command or similar command via computer 80, which in turn energizes exhaust fan assemblies 72, and thus exhaust fans 74. The “exhaust” command can be pre-programmed to run fans 74 for a selected period of time or can be manually terminated via a “stop exhaust” command or similar command on computer 80. When energized, fans 74 create a vacuum within the interior of housing 20 of decontamination and calibration station 10, which draws any residual alcohol reference standard gas and/or aerosolized disinfectant from the interior of the housing and/or the internal components of the connected vehicle ignition interlock device, such as the tubing or fuel cell, through the filters of exhaust fan assemblies 72, though the fans, and exhausts any residue to atmosphere.

In this manner, the exhaust feature of decontamination and calibration station 10 ensures that the interior of housing 20 and the internal components of the vehicle ignition interlock device are emptied of all residual alcohol reference standard gas and/or aerosolized disinfectant after calibration of the interlock device and/or sanitation and disinfectant of the interlock device and/or interior of the housing. After the calibrated and sanitized vehicle ignition interlock device is disconnected from decontamination and calibration station 10 and is returned to an operator, the exhaust feature of the station ensure that it is ready for use.

It is contemplated that exemplary embodiment vehicle ignition interlock device decontamination and calibration station 10 could include one or more sensors (not shown) integrated into or mounted within the decontamination and calibration station to monitor one or more processes of the decontamination and calibration station, such as during operation of the high-pressure purge feature, sanitization and disinfection feature, exhaust feature, and/or calibration of the vehicle ignition interlock device.

Thus, exemplary embodiment vehicle ignition interlock device decontamination and calibration station 10 of the disclosed subject matter overcomes the problems associated with and the deficiencies of prior art vehicle ignition device calibration systems and processes and includes a self-contained structure that enables a vehicle ignition interlock device to be operatively connected to the decontamination and calibration station within the structure via a modular cable and calibration tube of the station. Decontamination and calibration station 10 includes a high-pressure purge feature that ensures accurate calibration of the vehicle ignition interlock device and a clean sample chamber of the interlock device subsequent to calibration. Decontamination and calibration station 10 includes a sanitization and disinfecting feature that employs UV-C light bombardment or a disinfecting aerosol to sanitize and disinfect the interior of the station and the ignition interlock device prior to returning the device to a vehicle operator for use. Decontamination and calibration station 10 enables a vehicle ignition interlock device to be both calibrated and disinfected and sanitized with minimal interaction with the interlock device via a program for controlling and/or monitoring the processes, which is optionally operated by a remote computer or a computer integrated into the station, such as a simple touch screen interface operatively connected to the decontamination and calibration station and interlock device.

Accordingly, the exemplary embodiment vehicle ignition interlock device decontamination and calibration station of the disclosed subject matter is simplified, provides an effective, safe, inexpensive, and efficient structure which achieves all the enumerated objectives, provides for eliminating difficulties encountered with prior art vehicle ignition interlock device calibration stations, and solves problems and obtains new results in the art.

In the foregoing description, certain terms have been used for brevity, clarity and understanding; but no unnecessary limitations are to be implied therefrom beyond the requirements of the prior art, because such terms are used for descriptive purposes and are intended to be broadly construed. Moreover, the disclosed subject matter has been described with reference to a specific embodiment. It shall be understood that this illustration is by way of example and not by way of limitation, as the scope of the invention is not limited to the exact details shown or described. Potential modifications and alterations will occur to others upon a reading and understanding of the subject disclosure, and it is understood that the disclosed subject matter includes all such modifications and alterations and equivalents thereof.

Having now described the features, discoveries and principles of the disclosed subject matter, the manner in which the vehicle ignition interlock device decontamination and calibration station is constructed, arranged and used, the characteristics of the construction and arrangement, and the advantageous, new and useful results obtained; the new and useful structures, devices, elements, arrangements, parts and combinations are set forth in the appended claims.

What is claimed is:

1. A vehicle ignition interlock device decontamination and calibration station comprising:
a housing, said housing being configured to receive and enclose a vehicle ignition interlock device within an interior of the housing;
a valve assembly operatively and fluidly connected with said interior of said housing, said valve assembly being further operatively and fluidly connected to said vehicle ignition interlock device received and contained within the interior of the housing;
at least one reference standard being operatively connected to said valve assembly, said at least one reference standard enabling the ignition interlock device to be calibrated;
a decontamination feature integrated into said decontamination and calibration station, said decontamination feature enabling said decontamination and calibration station and/or said vehicle ignition interlock device received and contained in said interior of said housing to be sanitized and/or disinfected; and
a control system, said control system enabling actuation of the valve assembly and operation of the decontamination feature.

2. The vehicle ignition interlock device decontamination and calibration station of claim 1, wherein said decontamination and calibration station is self-contained.

3. The vehicle ignition interlock device decontamination and calibration station of claim 1, wherein said housing is a two-part housing that can be opened to access said interior of the housing and closed to seal said housing and enclose said vehicle ignition interlock device within the housing.

4. The vehicle ignition interlock device decontamination and calibration station of claim 3, wherein said two-part housing includes a top portion and a bottom portion, said top portion being pivotably connected to said bottom portion via a hinged connection, whereby the two-part housing is opened by upward arcuate movement of the top portion relative to the bottom portion to enable access to said interior.

5. The vehicle ignition interlock device decontamination and calibration station of claim 4, wherein said decontamination feature includes at least one UV-C bulb fixtures mounted on an inner surface of said top portion and at least one UV-C fixtures mounted on an inner surface of said bottom portion, said UV-C fixtures being operatively connected to said control system

6. The vehicle ignition interlock device decontamination and calibration station of claim 4, wherein said decontamination feature includes three UV-C bulb fixtures mounted on an inner surface of said top portion and three UV-C fixtures mounted on an inner surface of said bottom portion, said UV-C fixtures being operatively connected to said control system.

7. The vehicle ignition interlock device decontamination and calibration station of claim 1, wherein said decontamination feature includes at least one UV-C bulb fixture mounted within said interior of said housing.

8. The vehicle ignition interlock device decontamination and calibration station of claim 1, wherein said decontamination feature includes a cannister containing aerosolized disinfectant operatively connected to said valve assembly, actuation of the valve assembly via said control system enabling said aerosolized disinfectant to be introduced into said interior of said housing and/or said vehicle ignition interlock device received and contained within the interior of the housing.

9. The vehicle ignition interlock device decontamination and calibration station of claim 1, wherein said control system comprises a computer operatively connected to said decontamination and calibration station.

10. The vehicle ignition interlock device decontamination and calibration station of claim 9, wherein said computer is further operatively connected to said vehicle ignition interlock device when received and contained within said interior of said housing, the control system controlling calibration of the vehicle ignition interlock device with minimal interaction with said vehicle ignition interlock device.

11. The vehicle ignition interlock device decontamination and calibration station of claim 10, wherein said computer is a tablet attached to said housing.

12. The vehicle ignition interlock device decontamination and calibration station of claim 10, wherein said computer is operatively connected to said vehicle ignition interlock device via a modular data cable.

13. The vehicle ignition interlock device decontamination and calibration station of claim 1, wherein said control system includes a processor integrated into said decontamination and calibration station, said processor including a program for controlling functionally of at least said valve assembly and said decontamination feature.

14. The vehicle ignition interlock device decontamination and calibration station of claim 13, wherein said program is controllable from a remote device.

15. The vehicle ignition interlock device decontamination and calibration station of claim 1, said control system including a high-pressure purge feature, said high-pressure purge feature enabling said vehicle ignition interlock device to be purged by said at least one reference standard, the at least one reference standard being pressurized via said valve assembly.

16. The vehicle ignition interlock device decontamination and calibration station of claim 15, wherein said high-pressure purge feature is pre-programmable via said control system to enable purging of said vehicle ignition interlock device with said at least one reference standard for a selected period of time.

17. The vehicle ignition interlock device decontamination and calibration station of claim 1, further comprising an exhaust feature, said exhaust feature enabling said interior of said housing and/or said vehicle ignition interlock device to be exhausted to atmosphere.

18. The vehicle ignition interlock device decontamination and calibration station of claim 17, wherein said exhaust feature includes at least one exhaust fan assembly disposed within an opening formed in said housing, said at least one exhaust fan assembly being operatively connected to said control system.

19. The vehicle ignition interlock device decontamination and calibration station of claim 1, further comprising at least one sensor for monitoring one or more processes or conditions of the decontamination and calibration station.

20. A method for decontaminating and calibrating a vehicle ignition interlock device comprising the steps:
a. providing a decontamination and calibration station for a vehicle ignition interlock device;
b. enclosing said vehicle ignition interlock device within said decontamination and calibration station;
c. decontaminating the vehicle ignition interlock device via the decontamination and calibration station; and
d. calibrating said vehicle ignition interlock device with said decontamination and calibration station.
